(19) United States
(12) Patent Application Publication
(10) Pub. No.: US 2022/0330794 A1
(43) Pub. Date: Oct. 20, 2022

(54) ENDOSCOPE REPROCESSOR AND ACTUATION METHOD FOR ENDOSCOPE REPROCESSOR

(71) Applicant: OLYMPUS MEDICAL SYSTEMS CORP., Tokyo (JP)

(72) Inventors: Tomokazu IWASAKI, Tokyo (JP); Hideto ONISHI, Tokyo (JP)

(73) Assignee: OLYMPUS MEDICAL SYSTEMS CORP., Tokyo (JP)

(21) Appl. No.: 17/232,388

(22) Filed: Apr. 16, 2021

(51) Int. Cl. A61B 1/00

(52) U.S. Cl. CPC A61B 1/00119; A61B 1/00068; A61B 2562/0247

(57) ABSTRACT

An endoscope reprocessor includes: a pump; a port; a tube connected to the port; a connector disposed in the tube and connected to a pipe sleeve of a treatment instrument channel, which is a pipe of an endoscope, and configured to allow a fluid to be leaked from a connection region of the connector to the pipe sleeve to an outer circumferential surface of the pipe sleeve; a valve disposed in a flow path; a pressure sensor configured to measure a pressure of the fluid; and a processor configured to repeatedly control the valve such that the valve is closed when the pressure exceeds a first pressure and the valve is opened when the pressure is lower than a second pressure, and determine a feeding state of the fluid based on a time period until an opening/closing number of the valve reaches a predetermined number more than one.

### BACKGROUND OF I IIE INVENTION

### 1. Field of the Invention

The present invention relates to an endoscope reprocessor and an actuation method for the endoscope reprocessor. The endoscope reprocessor is an apparatus that performs reprocessing of an endoscope or endoscope accessories. The reprocessing is not particularly limited and may be rinsing by water, cleaning for removing dirt such as organisms_{—} disinfection for inactivating predetermined microorganisms, sterilization for eliminating or annihilating all microorganisms, or a combination of the cleaning, the disinfection, and the sterilization.

### 2pn. Description of the Related Art

Endoscopes are widely used in the medical field and the industrial field. In the endoscope used in the medical field, an insertion section is inserted into a body cavity to observe an inside of the body cavity and the insertion section is inserted into a treatment instrument channel, a pipe into which the insertion section is inserted, to perform treatment using a treatment instrument projecting from an opening at a distal end of the insertion section.

The endoscope is cleaned and disinfected using a reprocessor after being used. A connector of a. tube is connected to a pipe sleeve at an end portion of the pipe in order to clean and disinfect an inside of the pipe. A fluid for the cleaning and disinfection is supplied from the tube. The fluid is discharged from the opening at the distal end of the insertion section through the pipe.

International Publication No. 2009/146839 discloses a method of in a reprocessor in which a connector of a tube is connected to a pipe sleeve of a channel, detecting blockage of a pipe and a. connection failure of the connector.

International Publication No. 2018/168036 discloses a reprocessor that treats even an outer circumferential surface of a pipe sleeve of an endoscope by providing a gap, from which a fluid leaks out, in a connector of a tube.

### SUMMARY OF THE INVENTION

An endoscope reprocessor according to an embodiment includes: a pump configured to apply a pressure to a. fluid; at least one port from which the fluid discharged by the pump flows out; at least one tube having a first end connected to the at least one port; at least one connector disposed on a second end of the at least one tube and connected to a pipe sleeve of a pipe of an endoscope, the connector being configured to allow the fluid to be supplied to the pipe and to also allow the fluid to be leaked from a connection region between the connector and the pipe sleeve to an outer circumferential surface of the pipe sleeve; a valve disposed on a flow path of the fluid, the flow path being located between the pump and the port; a pressure sensor configured to measure a pressure of the fluid between the port and the valve; and a processor configured to repeatedly control the valve such that the valve is closed when the pressure exceeds a first pressure and the valve is opened when the pressure is lower than a second pressure, and determine a feeding state of the fluid based on a time period until an opening/closing number of the valve reaches a predetermined number more than one.

An endoscope reprocessor according to an embodiment includes: a pump configured to apply a pressure to a fluid; at least one port from which the fluid discharged by the pump flows out; at least one tube having a first end connected to the at least one port; at least one connector disposed on a second end of the at least one tube and connected to a pipe sleeve of a pipe of an endoscope, the connector being configured to allow the fluid to be supplied to the pipe and to also allow the fluid to be leaked from a connection region between the connector and the pipe sleeve to an outer circumferential surface of the pipe sleeve; a valve disposed on a flow path of the fluid, the flow path being located between the pump and the port; a pressure sensor configured to measure a pressure of the fluid between the port and the valve; and a processor configured to repeatedly control the valve such that the valve is closed in a first time period and the valve is opened in a second time period and determine a feeding state of the fluid based on a change in the pressure at a time when an opening/closing number of the valve is a predetermined plural number.

An actuation method for an endoscope reprocessor according to an embodiment includes: connecting, to a pipe sleeve of a pipe of an endoscope, a connector configured to supply a fluid to the pipe of the endoscope, the fluid leaking from a connection region of the connector to the pipe sleeve to an outer circumferential surface of the pipe sleeve, and connecting a first end of a tube, on a second end of which the connector is disposed, to a port to which the fluid is supplied; applying a pressure to the fluid using a pump; repeatedly controlling a valve disposed on a flow path of the fluid, the flow path being located between the pump and the port such that the valve is closed in a first time period and opened in a second time period; and determining a feeding state of the fluid based on a time period until an opening/closing number of the valve reaches a predetermined plural number.

### BRIEF DESCRIPTION OF THE DRAWINGS

FIG. 1 is an external view of an endoscope reprocessor in an embodiment;

FIG. 2 is a configuration diagram of the endoscope reprocessor in the embodiment;

FIG. 3 is a flowchart of operation of an endoscope reprocessor in a first embodiment:

FIG. 4A is a diagram for explaining the operation of the endoscope reprocessor in the first embodiment;

FIG. 4B is a diagram for explaining the operation of the endoscope reprocessor in the first embodiment;

FIG. 4C is a diagram for explaining the operation of the endoscope reprocessor in the first embodiment;

FIG. 5 is a flowchart of operation of an endoscope reprocessor in a modification of the first embodiment;

FIG. 6 is a diagram for explaining the operation of the endoscope reprocessor in the modification of the first embodiment;

FIG. 7 is a flowchart of operation of an endoscope reprocessor in a second embodiment;

FIG. 8A is a diagram for explaining the operation of the endoscope reprocessor in the second embodiment;

FIG. 8B is a diagram for explaining the operation of the endoscope reprocessor in the second embodiment; and

FIG. 8C is a diagram for explaining the operation of the endoscope reprocessor in the second embodiment.

### DETAILED DESCRIPTION OF THE PREFERRED EMBODIMENTS

### <Configuration of an Endoscope Reprocessor>

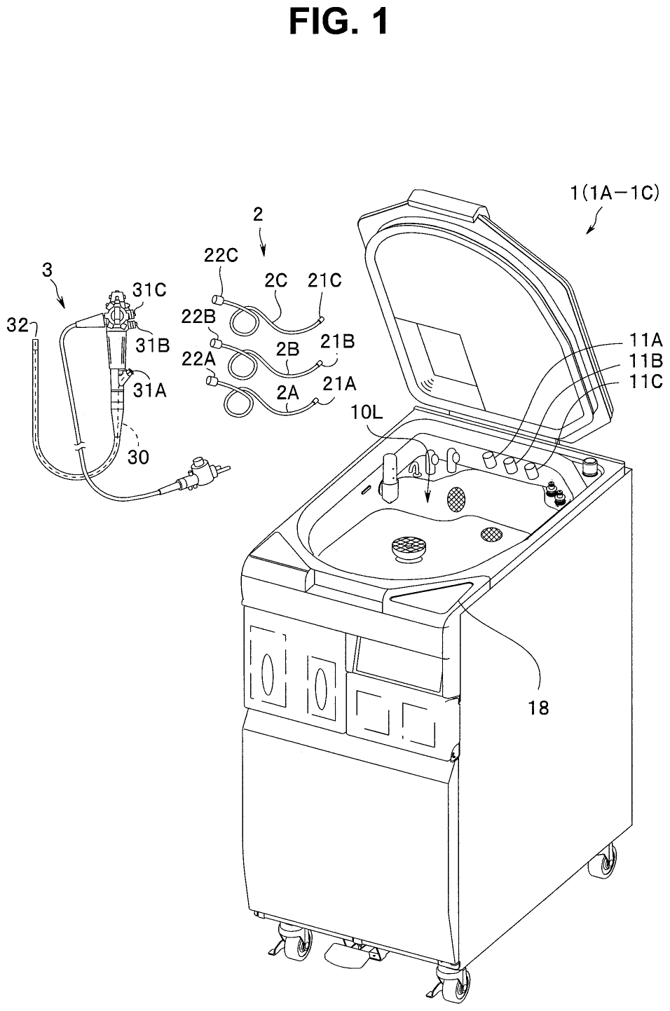

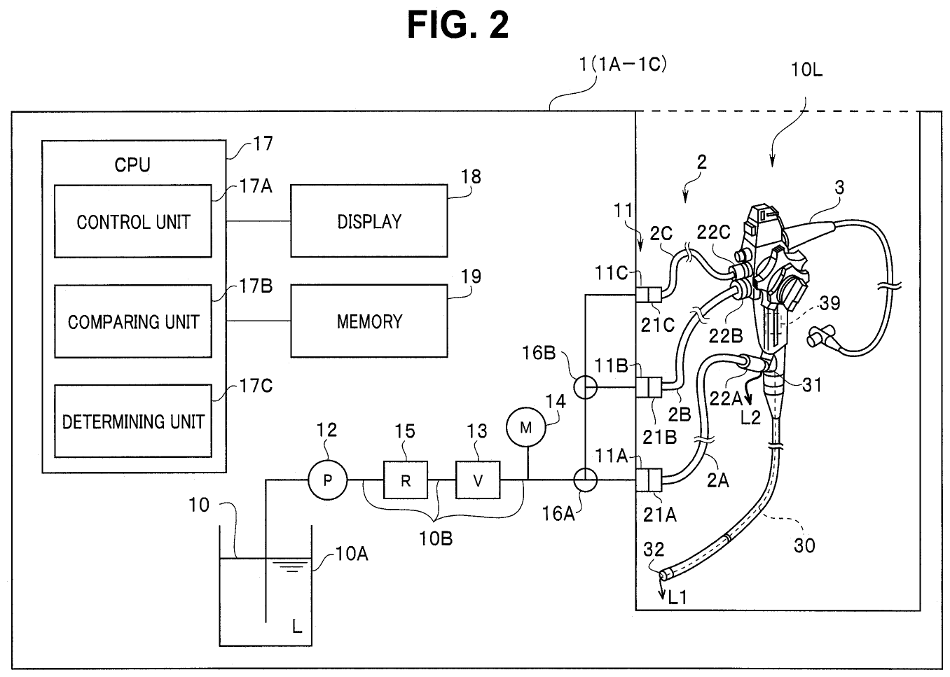

An endoscope reprocessor 1 (hereinafter referred to as “reprocessor 1”) in an embodiment shown in FIGS. 1 and 2 cleans a used endoscope 3 using cleaning liquid and disinfects the used endoscope 3 using disinfection liquid.

A treatment tank 10L, in which the endoscope 3 is housed, is provided in the reprocessor 1. When the endoscope 3 is treated, a fluid such as cleaning liquid is supplied to the treatment tank 10L. The reprocessor 1 includes, for example, on an upper surface, a display 18 that displays a treatment state and the like.

The endoscope 3 includes a treatment instrument channel 30, which is a pipe, an air/water feeding pipe (not shown), and a suction pipe (not shown). For example, a treatment instrument is inserted into the treatment instrument channel 30 from an operation section and projects from an opening 32 at a distal end portion of an insertion section.

In order to clean and disinfect an inner surface of the treatment instrument channel 30, a treatment instrument channel of the operation section and the reprocessor 1 are connected using a tube 2A. In other words, a connector 21,E disposed at a first end of the tube 2A is connected to a port 11A of the reprocessor from which the cleaning liquid or the like flows out. A connector 22A disposed on a second end of the tube 2A is connected to a pipe sleeve 31A of the treatment instrument channel 30 of the endoscope 3.

A connector 21B of a tube 2B is connected to a port 11B. A connector 22B of the tube 2B is connected to a pipe sleeve 31B of the air/water feeding pipe. A connector 21C of a tube 2C is connected to a port 11C. A connector 22C of the tube 2C is connected to a pipe sleeve 31C of the suction pipe. A fluid I, supplied from the ports 11A, 11B, and 11C flows into the pipe (for example, the treatment instrument channel 30) of the endoscope 3 through the tubes 2A, 2B, and 2C and, as shown in FIG. 2. flows out from the opening (for example, the channel opening 32) at the distal end portion (fluid L1).

Note that, in the following explanation, when each of a plurality of components having the same function is referred to, one character at an end of a sign is omitted. For example, each of the tubes 2A, 2B, and 2C is referred to as tube 2.

A case in which treatment is performed using the tube 2A and the cleaning liquid 10 is explained below as an example. As shown in FIG. 2, a part L2 of the fluid L leaks from a connection region of the connector 22A of the reprocessor 1 to the pipe sleeve 31A to an outer circumferential surface of the pipe sleeve 31A. Accordingly, the outer circumferential surface of the pipe sleeve 31A is also cleaned and disinfected.

In the connector 22, a groove provided in a contact portion with the pipe sleeve 31 and a through-hole leading from an inner surface to an outer surface are present. A fluid leaks through the groove and the through-hole. Note that the connector 22 may be configured such that the fluid leaks from the through-hole only when pressure of the fluid is low at a fluid supply start. For example, in a connector in which a partition wall of a flow path is urged by a spring, the partition wall moves and the fluid stops leaking from the through-hole when pressure of the fluid is larger than an urging force of the spring.

The reprocessor 1 includes tanks for the fluid L for cleaning and disinfection, for example, a tank 10A for the cleaning liquid 10 and a tank (not shown) for the disinfection liquid.

The cleaning liquid 10 in the tank 10A is sucked, applied with a pressure, and discharged by a pump 12. A regulator 15 that adjusts the cleaning liquid 10 to a predetermined pressure P0 is not an essential component. The cleaning liquid 10 discharged by the pump is supplied to the port 11A through a switching valve 16A. In other words, the switching valve 16A switches a flow path for the cleaning liquid 10 in a direction of one of the port I1A or a switching valve 16B. The switching valve 16B switches the flow path for the cleaning liquid 10 in a direction of one of the port 11B or the port 11C.

A valve 13 is disposed in a flow path 10B for the fluid 10 between the pump 12 and the port 11. A pressure sensor 14 measures pressure of the cleaning liquid 10 between the port 11 and the valve 13.

The fluid is supplied to any one of the ports 11A, 11B, and 11C according to the switching by the switching valves 16A and 16B.

The reprocessor 1 includes a processor which is a CPU (central processing unit) 17. For example, the CPU 17 reads a program stored in a memory 19 and operates. The program may be stored in a non-transitory computer-readable storage medium. Any one of a plurality of components of the CPU 17 may be configured by a dedicated hardware circuit.

A control unit 17A controls an overall operation of the reprocessor 1. As explained below, the valve 13, which is an on-off valve, repeats opening and closing according to control by the control unit 17A, A comparing unit 17B compares a predetermined value and a measurement value. A determining unit 17C determines a feeding state of the fluid based on a result of the comparing unit 17B.

The valve 13 and the pressure sensor 14 may be disposed in each of three flow paths between the switching valves 16A and 16B and the ports 11A to 11C. A primary side pipe of the pump 12 may be connected to a circulation port (not shown) provided on a bottom surface of the treatment tank 10L. In this case, the fluid U in the treatment tank 101, is circulated by the pump 12.

The fluid used for the determination may he disinfection liquid, tap water, or gas such as air instead of the cleaning liquid 10.

The endoscope 3 only has to include at least one pipe. Accordingly, the reprocessor 1 only has to include at least one port 11, at least one tube 2, and at least one connector 21.

Determination of blockage of the pipe and a connection failure of the connector is explained below. When it is determined that the pipe and the connector are normal, usual cleaning and disinfection treatment is performed.

### First Embodiment

The determining unit 17C of the reprocessor 1 in the present entbodiment determines a feeding state of a fluid based on a time period required for a plurality of opening and closing operations of the valve 13.

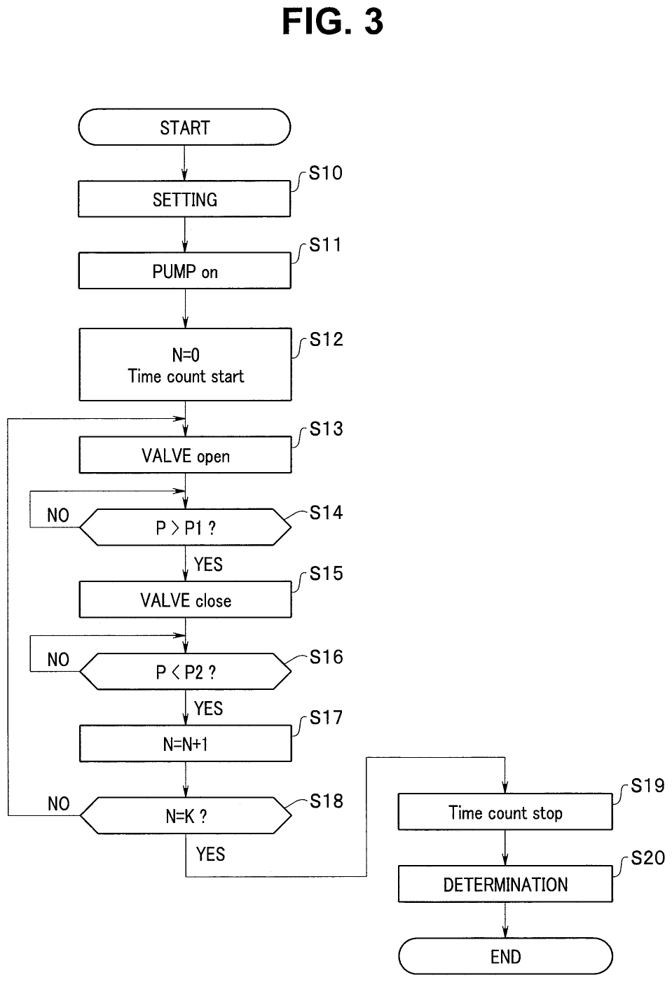

Operation of the reprocessor 1 is explained with reference to a flowchart of FIG. 3. The tube 2A is explained as an example below. In other words, the switching valve 16A is in a state in which the cleaning liquid 10 flows to the tube 2A.

### <Step S10> Setting

The connector 21A at the first end of the tube 2A is connected to the port 11A. The connector 22A at the second end of the tube 2A is connected to the pipe sleeve 31A of the treatment instrument channel 30.

### <Step S11> Pump ON

When the pump 12 is started according to the control by the control unit 17A, the cleaning liquid 10 from the tank 10A is applied with a pressure and supplied to the flow path 10B.

<Step S12> Initialization (N=0, Time count start)

The number N of an open/close cycle of the valve 13 is set to 0. For example, time measurement is started by a clock function of the control unit 17A.

### <Step S13> Valve Open

When the control unit 17A controls the valve 13 to an open state, the cleaning liquid 10 flows to the treatment instrument channel 30 through the port 11A and the tube 2A. The cleaning liquid 10 flows out from the opening 32 of the treatment instrument channel 30 and leaks from a gap of the connector 21A.

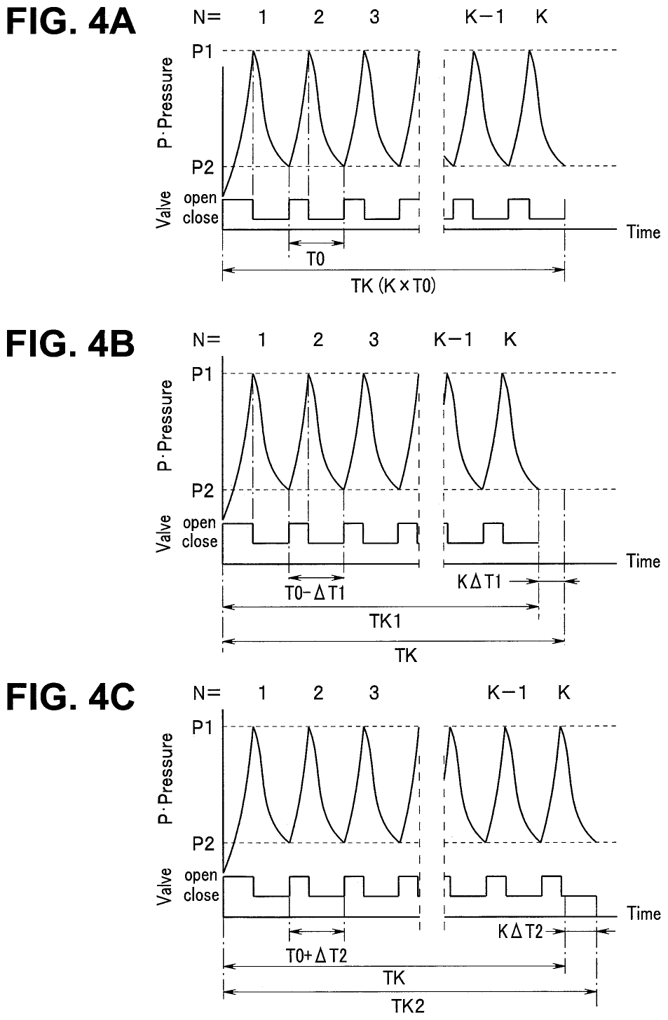

As shown in FIG. 4A, pressure P of the cleaning liquid 10 between the port and the valve 13 indicated by the pressure sensor 14 rises. The pressure P is different depending on not only a pressure loss in the treatment instrument channel 30 and the like but also a flow rate of the cleaning liquid 10 leaking from the connector 21A.

### <Step S14> P>P1?

The comparing unit 17B compares the pressure P of the pressure sensor 14 and a predetermined first pressure P1. The first pressure P1 is equal to or lower than pressure P0 adjusted by the regulator 15, for example, 50% of the pressure P0. When the pressure P is larger than the first pressure P (YES), the control by the control unit 17A shifts to step S15.

Note that when the pressure P is not larger than the predetermined first pressure P1 even if predetermined time, for example, 30 seconds elapse, the control unit 17A determines that the connector 21A or 22A is off, stops subsequent cleaning and disinfection treatment, and sounds an alarm.

### <Step S15> Valve Close

The control unit 17A controls the valve 13 to a closed state. As shown in FIG. 4A, since the cleaning liquid 10 flows out from the gap of the connector 21A and the opening 32 of the treatment instrument channel 30, the pressure P decreases.

### <Step S16> P<P2?

When the pressure P is smaller than a predetermined second pressure P2 (YES), the control by the control unit 17A shifts to step S17, For measurement accuracy and a reduction of a measurement time, the second pressure P2 is preferably, for example, less than 50% and more than 10% of the first pressure P1.

### <Step S17> N=N+1

1 is added to the number N of the open/close cycle of the valve 13.

<Step S18>N=K?

When the number N of the open/close cycle of the valve 13 reaches a predetermined number K (YES), the control by the control unit 17A shifts to step S19. The processing from step S13 is repeatedly performed until the number of N reaches the predetermined number K (NO). Accordingly, as shown in FIG. 4A, the pressure P repeats an increase and a decrease K times.

### <Step S19> Time Count Stop

The time measurement ends. in the example shown in FIG. 4A, a time period TS required for K cycles is K times a time period T0 of one open/close cycle.

### <Step S20> Determination

The determining unit 17C determines a feeding state of the cleaning liquid 10 based on the time period TS until the opening/closing number N of the valve 13 reaches a predetermined plural number K. When the feeding state is determined as normal, the cleaning and disinfection treatment is started.

FIG. 4A shows a pressure change in the case in which the feeding state is normal. A time period required for one open/close cycle is T0. In contrast, in an example shown in FIG. 4B, a time period required for one open/close cycle is (T0-ΔT1) shorter than T0. In the example shown in FIG. 4B, it is determined that connection of the connectors 21 and 22 is failure.

However, a time difference ΔT1 in one cycle is extremely small. Further, the time difference ΔT1 fluctuates at every cycle. Accordingly, it is likely that the determining unit 17C cannot perform an accurate determination based on the time difference ΔT1 in one cycle. In particular, in the connector 22 in which the fluid leaks from the connection region to the pipe sleeve 31 to the outer circumferential surface, accurate determination is not easy.

The reprocessor I determines the feeding state based on a time period TK1 until the opening/closing number N of the valve 13 reaches a predetermined plural number K. In other words, in FIG. 4A (normal), a time period required for K open/close cycles is a reference time TK. In contrast, in FIG. 4B (a connection failure), the time period TK1 required for the K open/close cycles is shorter than the reference time TK by a time difference KΔT. The time difference KΔT is a time difference obtained by accumulating, K times, the time difference ΔT1. in one cycle. Therefore, a clear difference occurs to make the determination easy.

The reference time TK is (TK=K×T0). Even if there is fluctuation in the time period TO required for one open/close cycle, since the reference time TK is a total in the K open/close cycles, time periods TOs are averaged. Even if there is fluctuation in the time difference ΔT1, since the time period TK1 is also a total in the K open/close cycles, the time period TK1 is averaged.

On the other hand, in an example shown in FIG. 4C, a time period TK2 required for the K open/close cycles is (TK+KΔT2) longer than the reference time TK. In the example shown in FIG. 4C, it is determined that the treatment instrument channel 30, which is the pipe of the endoscope 3, is blocked.

Even if a time difference ΔT2 is very small and there is fluctuation in the time difference ΔT2, since the time period TK2 is a total in the K open/close cycles, a clear difference from the reference time TK occurs and the time period TK2 is averaged.

Note that the predetermined number K is more than one but is preferably more than 5 and less than 100. If the predetermined number K is larger than the range, determination is accurate. If the predetermined number K is smaller than the range, quick determination can be performed. Note that the K open/close cycles may be performed a plurality of times and a result of the plurality of times of the K open/close cycles may be averaged to perform determination.

The reprocessor 1 includes the connector 22 in which a fluid leaks from the connection region to the pipe sleeve 31 to the outer circumferential surface, A feeding state of the fluid can be accurately determined. For example, when a time period required for the K open/close cycles is smaller than 90% of the reference time TK and when the time period is larger than 110% of the reference time TK, the determining unit 17C determines that the feeding state is “abnormal”. When a determination result of the determining unit 17C is “normal”, usual clearing and disinfection treatment is performed. When the determination result is “abnormal”, the control unit 17A preferably stops the treatment and displays “abnormal” on the display 18 or emits alarm sound to notify a user. Timing for performing a determining operation is preferably timing of start of feeding of each of cleaning liquid, disinfection liquid, rinsing water, and the like but may be a start time of feeding of a part of the cleaning liquid, disinfection liquid, rinsing water, and the like.

Note that, as explained above, the endoscope 3 includes the plurality of pipes (for example, the treatment instrument channel 30, the water/air feeding pipe, and the suction pipe). Therefore, the cleaning liquid 10 is fed to the respective pipes to determine the feeding state, Accordingly, the reprocessor 1 includes a plurality of ports 11A to 11C, a plurality of tubes 2A to 2C, and a plurality of connectors 22A to 22C.

The CPU 17 preferably sets, according to a specification of the endoscope and a specification of the plurality of pipes, measurement conditions such as the first pressure P1, the second pressure P2, the reference time 1K, and the predetermined number K and determines feeding states under the respective measurement conditions.

For example, in an endoscope for large intestine test having a long insertion section and a nasal endoscope having a thin pipe because an insertion section has a small diameter, measurement conditions different from measurement conditions of a peroral endoscope for stomach test are set. Different measurement conditions are respectively set for the treatment instrument channel 30, the air/water feeding pipe, and the suction pipe.

The measurement conditions of the reprocessor 1 and the reference time TK are selected by the user before a treatment start from conditions and the like stored in advance in the memory 19 of the reprocessor 1. Note that when the endoscope 3 includes a memory 39 or identifying means such as a barcode, condition setting is easy.

### <Modification of the First Embodiment>

A reprocessor 1A in the modification is similar to the reprocessor 1 in the first embodiment. Therefore, components having the same functions are denoted by the same reference numerals and signs and explanation of the components is omitted.

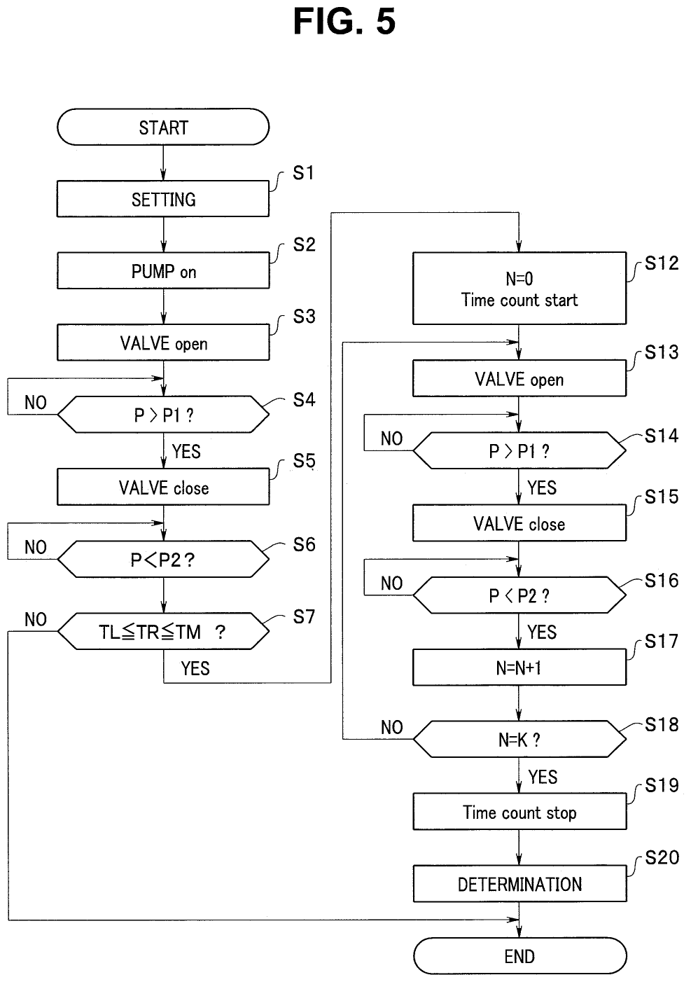

Operation of the reprocessor IA is explained with reference to a flowchart of FIG. 5.

### <Steps S1 to S6>

Since steps S1 to S6 are the same as steps S10 to S 16 of the reprocessor 1 explained with reference to FIG. 3, explanation about steps S1 to S6 is omitted.

### <Step S7> TL≤TR≤TM?

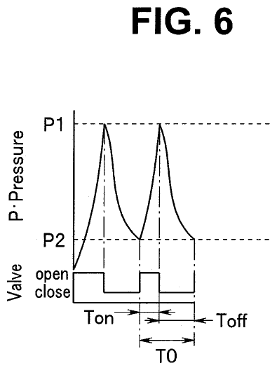

As shown in FIG. 6, when opening and closing of the valve 13 is controlled based on the pressure P, the time period T0 of one cycle is a sum of a time period Ton in which the valve 13 is open and a time period Toff in which the valve 13 is closed. The time period Ton and the time period. Toff are measured by, for example, the clock function of the control unit 17A.

A ratio TM (TM=Ton/Toff) of the time period Ton in which the valve is open and the time period Toff in which the valve is closed changes compared with a normal time if the connector 22 is off from the pipe sleeve 31. For example, when a ratio TR (TR=measurement TM/normal TM) is smaller than a predetermined minimum ratio TL or larger than a predetermined maximum ratio TM, it is highly likely that some abnormality has occurred.

For example, the minimum ratio TL is 0.7 and the maximum ratio TM is 1.3.

In the reprocessor 1A, the comparing unit 17B of the CPU 17 performs comparison using the ratio TR in an initial period of cycle treatment, for example, the ratio TR in one cycle or the ratio TR in two cycles in which operation is stable. In case of (TL≤TR≤TM), that is, (TR<TL) or (TR>TM), the CPU 17 stops subsequent treatment. When it is not determined that the feeding state is abnormal, usual treatment in the plurality of steps S12 to S20 is performed. If it is determined that the feeding state is normal, the cleaning and disinfection treatment is started.

The reprocessor 1A can determine abnormality in a short time based on the ratio TM (Ton/Toff) of the time period Ton in which the valve is open and the time period Toff in which the valve is closed.

Note that when it is not determined, based on the ratio TM (Ton/Toff) of the time period Ton in which the valve is open and the time period Toff in which the valve is closed, that the feeding state is abnormal, the reprocessor 1A may start the cleaning and disinfection treatment without performing the usual treatment in the plurality of steps S12 to S20.

### Second Embodiment

A reprocessor 1B in the present embodiment is similar to the reprocessor 1 in the first embodiment. Therefore, components having the same functions are denoted by the same reference numerals and signs and explanation of the components is omitted.

The determining unit 17C of the reprocessor 1B in the present embodiment determines a feeding state of the fluid L based on a change in the pressure P at a time when the opening/closing number N of the valve 13, which repeats opening and closing at a constant time period, reaches the predetermined plural number K.

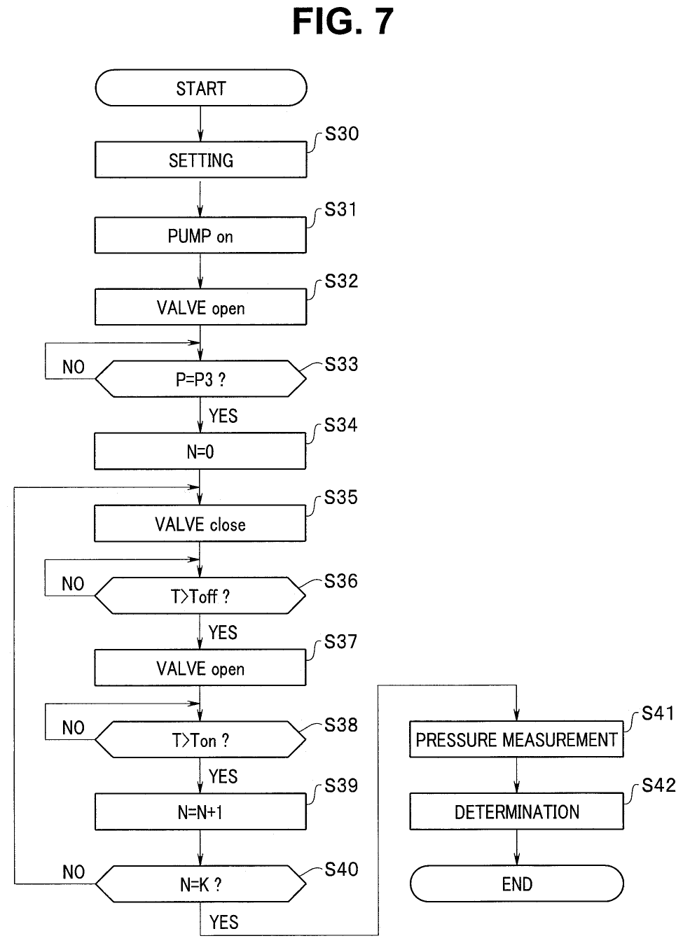

Operation of the reprocessor 1B is explained with reference to a flowchart of FIG. 7.

### <Steps S30 to S32>

Since steps S30 to S32 are the same as steps S10, S11, and S12 explained above, explanation about steps S30 to S32 is omitted.

### <Step S33> P=P3?

When the pressure P reaches a predetermined initial pressure P3 (YES), the control by the control unit 17A shifts to step S34. Note that when the pressure P does not reach the predetermined initial pressure P3 even if predetermined time, for example, 30 seconds elapse, the CPU 17 stops subsequent cleaning and disinfection treatment and sounds an

### <Step S34> N=0

The number of N of the open/close cycle of the valve 13 is initialized.

### <Step S35> Valve Close

The control unit 17A controls the valve 13 to a closed state. As shown in FIG. 5A, the cleaning liquid 10 flows out from the gap of the connector 21A and the opening 32 of the treatment instrument channel 30, which is the pipe. Therefore, the pressure P decreases.

### <Step S36> T>Toff?

When a predetermined close time period Toff elapses (YES), the control by the control unit 17A shifts to step S37.

### <Step S37> Valve Open

The control unit 17A controls the valve 13 to an open state. As shown in FIG. 5A. the pressure P rises.

### <Step S38> T>Ton?

When a predetermined open time period Ton elapses (YES), the control by the control unit 17A shifts to step S39.

### <Step S39> N=N+1

1 is added to the number of N of the open/close cycle of the valve 13.

### <Step S40> N=K?

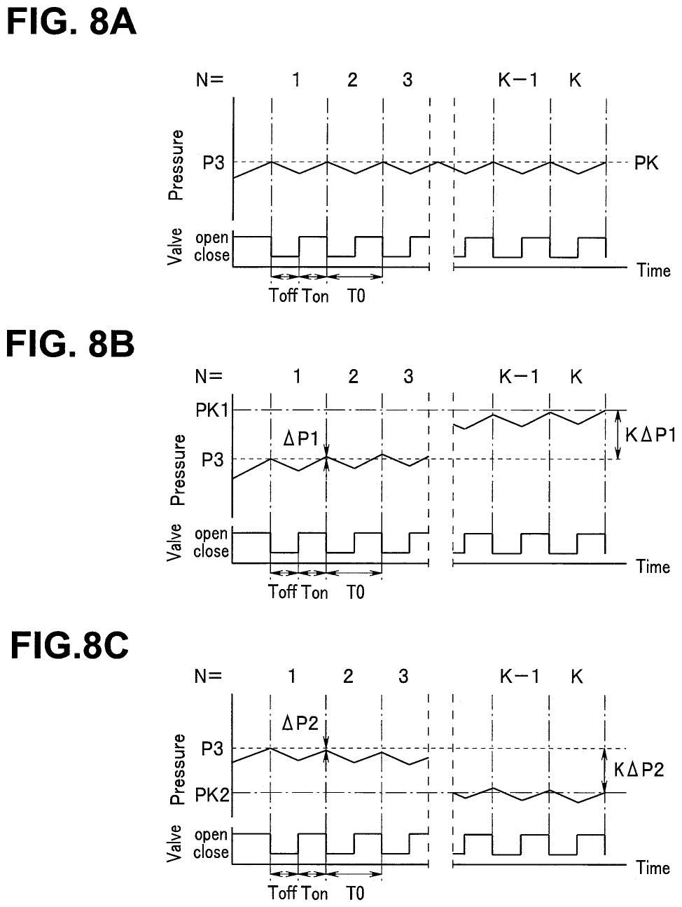

When the number of N of the open/close cycle of the valve 13 reaches the predetermined number K (YES), the control by the control unit 17A shifts to step S41. The processing from step S35 is repeatedly performed until the number of N reaches the predetermined number K (NO). Accordingly, as shown in FIG. 8A, the pressure P repeats an increase and a decrease K times.

### <Step S41> Pressure Measurement

The pressure P of the fluid is measured by the pressure sensor 14.

### <Step S42> Determination

The determining unit 17C determines a feeding state of the cleaning liquid 10 based on the pressure PK at a time when the opening; closing number of the valve 13 is the predetermined plural number K. If it is determined that the feeding state is normal, the cleaning and disinfection treatment is started.

FIG. 8A shows a pressure change in the case in which the feeding state is normal. The pressure PK is substantially the same with respect to the initial pressure P3. In other words, the open/close cycle is set such that the pressure P does not change at a normal time.

In contrast, in an example shown in FIG. 8B, the pressure P increases by ΔP1 after one open/close cycle. In the example shown in FIG. 8B, it is determined that the pipe of the endoscope is blocked. In an example shown in FIG. 8C, the pressure P decreases by ΔP2 after one open/close cycle. In the example shown in FIG. 8C, it is determined that a connection failure of the connectors 21 and 22 occurs.

However, a pressure difference ΔP1 in one cycle is extremely small. Further, the pressure difference ΔP1 fluctuates at every cycle. Accordingly, it is likely that an accurate determination cannot be performed based on the pressure difference ΔP1 in one cycle. In particular, in the connector 22 in which the fluid leaks from the connection region to the pipe sleeve 31 to the outer circumferential surface, accurate determination is not easy.

The reprocessor I determines the feeding state based on the pressure P at a time when the opening/closing number N of the valve 13 is the predetermined plural number K. In other words, in FIG. 8A (normal), the pressure P after the K open/close cycles is the same reference pressure PK as the initial pressure P3 with respect to the initial pressure P3. In contrast, in FIG. 8B (blockage), a pressure PK1 after the K open/close cycles increases from the initial pressure P3 by a pressure difference KΔP1. The pressure difference KΔP1 is a pressure difference obtained by accumulating, K times, the pressure difference SP 1 in one cycle. Therefore, a. clear difference from the reference pressure PK occurs and the pressure difference KΔP1 is averaged to make the determination easy.

Even if there is fluctuation in the pressure change ΔP1 in one open/close cycle, since the pressure PK1 is a total in the K open/close cycles, a pressure change is averaged.

On the other hand, in FIG. 8C (a connection failure), a pressure PK2 after the K open/close cycles decreases from an initial pressure P3 by KΔP2.

Even if a pressure difference ΔP2 is very small and there is fluctuation, since the pressure PK2 is also a total in the K open/close cycles, a clear difference from the reference pressure PK occurs and the pressure PK2 is averaged.

The time period T0 of one cycle is preferably longer than 5 seconds and shorter than 60 seconds. The predetermined number K is more than one but is preferably more than 5 and less than 100. If both of the time period T0 and the predetermined number K are larger than the ranges, the determination is accurate. If both of the time period T0 and the predetermined number K are smaller than the ranges, quick determination can be performed. Note that a plurality of times of the K open/close cycles may be performed and a result of the plurality of times of the K open/close cycles may be averaged to perform the determination.

The reprocessor 1B includes the connector 22 in Which a fluid leaks from the connection region to the pipe sleeve 31 to the outer circumferential surface. A feeding state of the fluid can be accurately determined. For example, when pressure after the K open/close cycles is smaller than 90% of the reference pressure PK and when the pressure after the K open/close cycles is larger than 110% of the reference pressure PK, the determining unit 17C determines that the feeding state is “abnormal”. The control unit 17A stops subsequent cleaning and disinfection treatment and sounds an alarm.

Note that, in the reprocessor 1B, the determination is performed based on the pressure change after the pressure P reaches the initial pressure P3. However, the determination may be performed based on a pressure change from a state in which the initial pressure is zero.

In the embodiments and the modifications explained above, a medical flexible endoscope is explained as an example. However, the endoscope in the embodiment of the present invention may be an industrial endoscope, a rigid endoscope, or a wireless endoscope without a universal cord.

The present invention is not limited to the embodiments explained above. Various changes, alternations, and the like are possible in a range in which the gist of the invention is not changed.

What is claimed is:

1. An endoscope reprocessor comprising:
a pump configured to apply a pressure to a fluid;
at least one port from which the fluid discharged by the pump flows out;
at least one tube having a first end connected to the at least one port;
at least one connector disposed on a second end of the at least one tube and connected to a pipe sleeve of a pipe of an endoscope, the connector being configured to allow the fluid to be supplied to the pipe and to also allow the fluid to be leaked from a connection region between the connector and the pipe sleeve to an outer circumferential surface of the pipe sleeve;
a valve disposed on a flow path of the fluid, the flow path being located between the pump and the port;
a pressure sensor configured to measure a pressure of the fluid between the port and the valve; and
a processor configured to repeatedly control the valve such that the valve is closed when the pressure exceeds a first pressure and the valve is opened when the pressure is lower than a second pressure, and determine a feeding state of the fluid based on a time period until an opening/closing number of the valve reaches a predetermined number more than one.

2. The endoscope reprocessor according to claim 1, wherein the predetermined number is 5 or more and 100 or less.

3. The endoscope reprocessor according to claim 1. further comprising,
a plurality of ports, a plurality of tubes, and a plurality of connectors, wherein
the endoscope includes a plurality of pipes, and
the processor sets the first pressure, the second pressure, and the predetermined number and determines the feeding state, according to a specification of the endoscope and a specification of the pipe.

4. The endoscope reprocessor according to claim 1, wherein, before determining the feeding state of the fluid based on the time until the opening/closing number of the valve reaches the predetermined number, the processor determines the feeding state of the fluid based on a ratio of a time period in which the valve is closed and a time period in which the valve is opened.

5. An endoscope reprocessor comprising:
a pump configured to apply a pressure to a fluid;
at least one port from which the fluid discharged by the pump flows out;
at least one tube having a first end connected to the at least one port;
at least one connector disposed on a second end of the at least one tube and connected to a pipe sleeve of a pipe of an endoscope, the connector being configured to allow the fluid to be supplied to the pipe and to also allow the fluid to be leaked from a connection region between the connector and the pipe sleeve to an outer circumferential surface of the pipe sleeve;
a valve disposed on a flow path of the fluid, the flow path being located between the pump and the port;
a pressure sensor configured to measure a pressure of the fluid between the port and the valve; and
a processor configured to repeatedly control the valve such that the valve is closed in a first time period and the valve is opened in a second time period and determine a feeding state of the fluid based on a change in the pressure at a time when an opening/closing number of the valve is a predetermined plural number.

6. The endoscope reprocessor according to claim 5, wherein
the first time period and the second time period are 5 seconds or more and 60 seconds or less, and
the predetermined number is 5 or more and 100 or less.

7. The endoscope reprocessor according to claim 5, further comprising,
a plurality of ports, a plurality of tubes, and a plurality of connectors, wherein
the endoscope includes a plurality of pipes, and
the processor sets the first time period, the second time period, and the predetermined number and determines the feeding state, according to a specification of the endoscope and a specification of the plurality of pipes.

8. An actuation method for an endoscope reprocessor, comprising:
connecting, to a pipe sleeve of a pipe of an endoscope, a connector configured to supply a fluid to the pipe of the endoscope, the fluid leaking from a connection region of the connector to the pipe sleeve to an outer circumferential surface of the pipe sleeve, and connecting a first end of a tube, on a second end of which the connector is disposed, to a port to which the fluid is supplied;
applying a pressure to the fluid using a pump;
repeatedly controlling a valve disposed on a flow path of the ⁻fluid, the flow path being located between the pump and the port such that the valve is closed in a first time period and opened in a second time period; and
determining a feeding state of the fluid based on a time period until an opening/closing number of the valve reaches a predetermined plural number.
